(19) United States
(12) Patent Application Publication
(10) Pub. No.: US 2003/0111081 A1
(43) Pub. Date: Jun. 19, 2003

(54) Detachable nasal cannula assembly

(76) Inventor: Parshotam C. Gupta, Grafton, OH (US)

Correspondence Address: Mark E. Bandy, Esq., Fay, Sharpe, Fagan, Minnich & McKee, LLP, 1100 Superior Avenue, Seventh Floor, Cleveland, OH, 44114-2579, US

(21) Appl. No.: 10/268,423

(22) Filed: Oct. 10, 2002

Related U.S. Application Data
(60) Provisional application No. 60/341,923, filed on Dec. 19, 2001.

(51) Int. Cl. A61M 15/08

(52) U.S. Cl. 128/207.18

(57) ABSTRACT

A detachable nasal cannula assembly is described that provides oxygen to a patient or delivers carbon dioxide from a patient to a monitor for testing. A nasal and head portion of the cannula assembly is detachable from a main tubing that is connected to an oxygen supply or a carbon dioxide monitor. The detachable nasal cannula assembly provides significant cost savings over presently known nasal cannulae.

### CROSS REFERENCES TO RELATED APPLICATIONS

The present application claims priority from U.S. provisional application serial No. 60/341,923, filed on Dec. 19, 2001.

### FIELD OF THE INVENTION

The present invention is directed to a detachable nasal cannula assembly adapted to provide oxygen to a patient or deliver carbon dioxide from a patient to a monitor for testing. The nasal and head portion of the cannula assembly is detachable from the main tubing. The nasal cannula assembly provides significant cost savings over currently known nasal cannulae.

### BACKGROUND OF THE INVENTION

Nasal cannulae are known and widely used in the field of medicine. For example, U.S. Pat. No. 4,106,505 to Salter et al., U.S. Pat. No. 5,188,101 to Tumedo, U.S. Pat. No. 5,271,391 to Gewes, U.S. Pat. No. 5,526,806 to Sansomi, and U.S. Pat. No. 5,509,409 to Weatherbelt, all of which are hereby incorporated by reference, each relate generally to nasal cannulae. Nasal cannulae are used in the medical environment to supply oxygen to a patient or provide a carbon dioxide sample from the patient. In general, the nasal cannula is connected to a patient so that the nasal projections from the nasal cannula are inserted into the nasal passageway of a patient. A nasal cannula may provide oxygen from an oxygen supply to a patient. Alternatively, a nasal cannula may provide a sample of carbon dioxide exhaled by the patient to a suitable carbon dioxide monitor.

Presently, nasal cannulae used in the medical environment must be entirely disposed of after its use by a patient. The disposal of nasal cannulae includes the tubing portion that is inserted into the nostrils and that which extends around the head of a patient, and the tubing portion connected to the oxygen source or carbon dioxide monitor. Unfortunately, the disposal of nasal cannulae in such a fashion is not cost beneficial. The nasal cannula disposed for each patient is costly because of the amount of tubing of the nasal cannulae that must be discarded after each use. Also, during its disposal nasal cannulae must be treated as a biohazard, so that special costly precautions must be exercised for its removal. Thus, the greater the amount of nasal cannulae tubing discarded, the greater the costs involved in its disposal.

It would be beneficial to dispose a smaller overall portion of the nasal cannula in order to increase the amount of cost savings. It would also be beneficial to reuse a major portion of the cannula assembly in order to minimize the amount of biohazard waste.

### SUMMARY OF THE INVENTION

The present invention is directed to a detachable nasal cannula assembly designed for use with an oxygen source and/or a carbon dioxide monitor. The detachable nasal cannula assembly has at least one, and preferably two channels suited for administering oxygen into the patient or withdrawing carbon dioxide from the patient.

The tubing assembly is modular and includes one or more tubing connectors releasably connecting a nasal cannula portion to a main tubing portion between the ends of the tubing. The tubing connectors allow for the separation of the nasal cannula portion, which is inserted into the nasal passageway of a patient, from the main tubing portion connected to an oxygen source or carbon dioxide monitor. The nasal cannula portion is disposed after each use, while the main tubing can be used numerous times before its disposal. The assembly can include one or more clamps that selectively close off one or both of the tubing channels.

The detachable cannula assembly allows a portion of the main tubing that is connected to the oxygen source or carbon dioxide monitor to be retained and reused by connection to a different nasal cannula portion. Only the nasal cannula portion itself that is in contact with the patient must be replaced. The use of the detachable cannula assembly should lead to cost savings since a major portion of the cannula assembly, i.e., the main tubing portion, may be reused. Additionally, lesser amounts of biohazard waste are created since only a minor portion of the cannula tubing assembly, i.e. the nasal cannula, must be discarded after each use.

The present invention provides, in a first aspect, a detachable nasal cannula assembly comprising a tubular nasal cannula portion having a first open end, a second open end opposite from the first end, a first nasal projection providing access to the interior of the tubular portion, a second nasal projection also providing access to the interior of the tubular portion, and a diaphragm disposed between the first and the second nasal projection. The diaphragm precludes communication between the first nasal projection and the second nasal projection through the nasal cannula portion. The detachable nasal cannula assembly also comprises a first main tubing portion having a first end and a second end opposite from the first end. One or both of the first and second ends of the first main tubing portion are releasably engageable with the first open end of the nasal cannula portion. The detachable nasal cannula assembly further comprises a second main tubing portion also having first and second opposite ends. One or both of the ends of the second main tubing portion are releasably engageable with the second end of the nasal cannula portion.

In another aspect, the present invention provides a detachable nasal cannula assembly comprising a tubular nasal cannula portion having a first open end, a second open end opposite from the first end, a first nasal projection providing access to the interior of the tubular portion, a second nasal projection also providing access to the interior of the tubular portion, and a diaphragm disposed between the first and the second nasal projection. The diaphragm precludes communication between the first nasal projection and the second nasal projection through the nasal cannula portion. The detachable nasal cannula assembly also comprises a main tubing component including a first end having a first interconnecting portion adapted to releasably engage the first open end of the nasal cannula portion and a second interconnecting portion adapted to releasably engage the second end of the nasal cannula portion. The main tubing component further includes a second end opposite from the first end.

In yet another aspect, the present invention provides a detachable nasal cannula assembly comprising a nasal tubular portion including a first nasal projection, a second nasal projection, an interconnecting portion, a first tubular segment extending between the first nasal projection and the interconnecting portion, and a second tubular segment extending between the second nasal projection and the interconnecting portion. The detachable nasal cannula assembly also comprises a main tubing component having a first end and a second end opposite from the first end. The first end is adapted to releasably engage the interconnecting portion of the nasal tubular portion.

Other features and benefits of the present invention will come to light in reviewing the following written specification and accompanying drawings.

### BRIEF DESCRIPTION OF THE DRAWINGS

The foregoing and other objects, features and advantages of the present invention should become apparent in the following description when taken in conjunction with the accompanying drawings, in which:

FIG. 1 is a perspective view illustrating the positioning of a preferred embodiment nasal cannula assembly on the face of a patient and connected to an oxygen supply and carbon dioxide monitor.

FIG. 2A is a front view illustrating a nasal cannula portion separate from the main tubing, of the preferred embodiment assembly.

FIG. 2B is a front view illustrating the main tubing separate from the nasal cannula portion, of the preferred embodiment assembly.

FIG. 3 is a front view illustrating an alternate preferred embodiment detachable nasal cannula assembly having one main tubing.

FIG. 4 is a front view illustrating another preferred embodiment nasal cannula assembly that simultaneously supplies oxygen and removes carbon dioxide.

FIG. 5 is a front view illustrating yet another preferred embodiment nasal cannula assembly supplying oxygen.

### DETAILED DESCRIPTION OF THE PREFERRED EMBODIMENTS

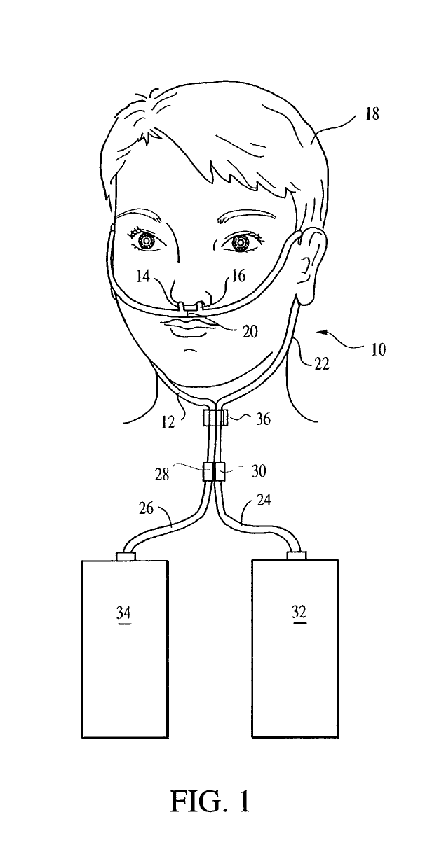

FIG. 1 illustrates, in accordance with the present invention, a preferred embodiment detachable nasal cannula assembly, generally designated as 10, comprising a tubular nasal cannula portion 22 and main tubing portions 24 and 26. The nasal portion 22 includes a hollow tubular body 12 and has two nasal projections 14 and 16, each extending outwardly and adapted to fit within a corresponding nasal passage of the nose of a patient 18. The nasal projections provide access to the interior of the tubular nasal cannula portion 22. The assembly 10 is held on the face of a patient 18 by looping the assembly 10 over the ears of the patient 18, although any other known means for holding the assembly 10 on the face of the patient 18, such as adhesive tape (not shown), can be used. A diaphragm 20 acts as a barrier separating the hollow body 12 of the assembly 10 into an inhalation portion, where for instance, oxygen is supplied to a patient from an oxygen supply, and an exhalation portion, where for example, carbon dioxide flows from the patient to a carbon dioxide monitor. One of the nasal projections 14 or 16 can serve as the inhalation portion, and the other nasal projection can serve as the exhalation portion. Alternatively, both nasal projections 14 and 16 can act as the inhalation portion or exhalation portion simultaneously.

The outer diameter of the nasal projections 14 and 16 is generally smaller than the opening of the nasal passage of a patient 18 so that the projections can be at least partially inserted into the nasal passage.

As noted, the assembly 10 comprises a nasal cannula portion 22 and main tubing portions 24 and 26. The nasal cannula portion 22 is releasably secured to the main tubing portions 24 and 26 by tubing connectors 28 and 30. Main tubing portion 24 is connected to a carbon dioxide monitor 32 and main tubing portion 26 is connected to an oxygen source 34. However, main tubing portion 24 can be connected to the oxygen source 34, while main tubing portion 26 can be connected to the carbon dioxide monitor 32. Alternatively, both main tubing portions 24 and 26 can be connected to the carbon dioxide monitor 32 or the oxygen source 34. The assembly 10 can therefore conduct both oxygen supplementation and carbon dioxide sampling simultaneously, or conduct either oxygen supplementation or carbon dioxide sampling. The assembly 10 may further include a clip 36 or other closure device that clamps the hollow body 12 and restricts the flow of oxygen or carbon dioxide (or any gas) in one or both of the main tubings 24 and 26 or one or both portions of the nasal cannula portion 22. The clip 36. is preferably retained with or generally secured to the nasal portion 22 and preferably near one or both of the ends 38 and 40.

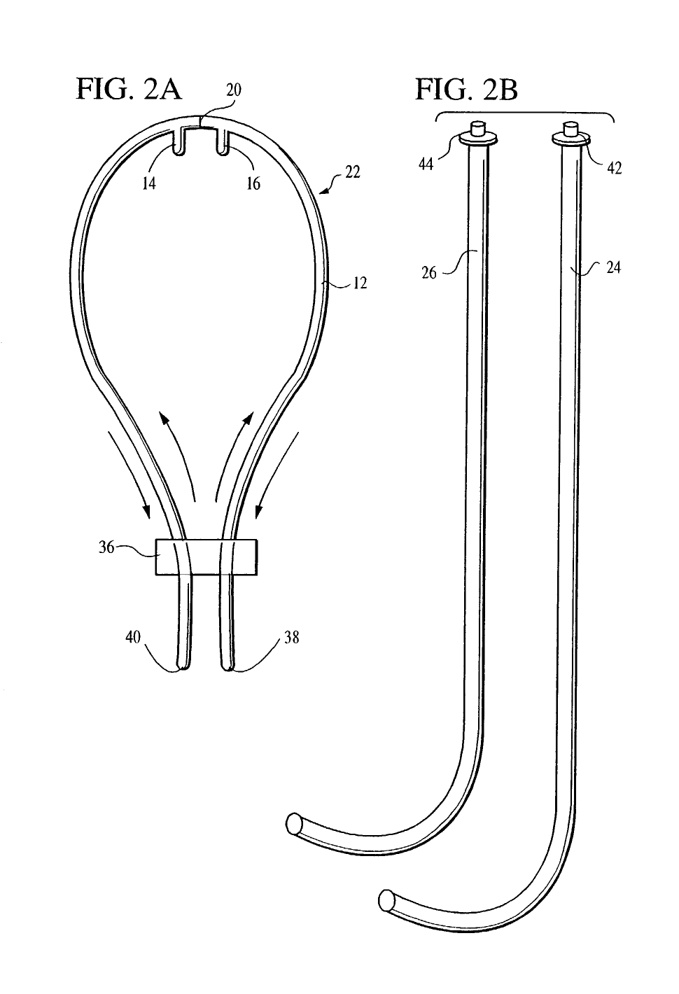

FIGS. 2A and 2B collectively illustrate the preferred embodiment detachable cannula assembly, where the nasal cannula portion 22 is detached from the main tubing 24 and 26. In particular, FIG. 2A shows the nasal cannula portion 22, and FIG. 2B shows main tubing portions 24 and 26. The ends of the nasal cannula portion 22 include interconnecting end portions 38 and 40. The main tubing portion 24 includes an interconnecting portion 42 that is adaptable to the interconnecting portion 38, and main tubing 26 includes an interconnecting portion 44 that is adaptable to the interconnecting portion 40. When the interconnecting portion 42 is connected to interconnecting portion 38 and interconnecting portion 44 is connected to interconnecting portion 40, tubing connections 28 and 30, as shown in FIG. 1, are formed. The mode of connection in connecting interconnecting portions 42 and 44 with interconnecting portions 38 and 40 include female/male connection, screw fastening, and any other known releasably fastenable mode of connection. The interconnecting portions 38, 40, 42 and 44 may also be in the form of nearly any shape so that the ends of the main tubing 24 and 26 are releasably fastenable with the ends 38 and 40 of the nasal cannula portion 22. The arrows shown in FIG. 2A illustrate the possible flow direction of gas within the nasal cannula portion 22.

By way of example, and not by way of limitation, the nasal cannula portion 22 can have an overall length of from about 15 to about 30 inches, preferably about 24 inches, although any length is suitable. The noted length being the distance measured along the tubular body 12 between ends 38 and 40. The length of the main tubing 24 and 26 can be from about 75 inches to about 100 inches, more preferably about 96 inches, although the length can vary, as that noted length being the distance as measured along the main tubing portions 24 or 26, between either of the ends 42 or 44 and a corresponding end at which the oxygen source 34 or the carbon dioxide monitor 32 is connected.

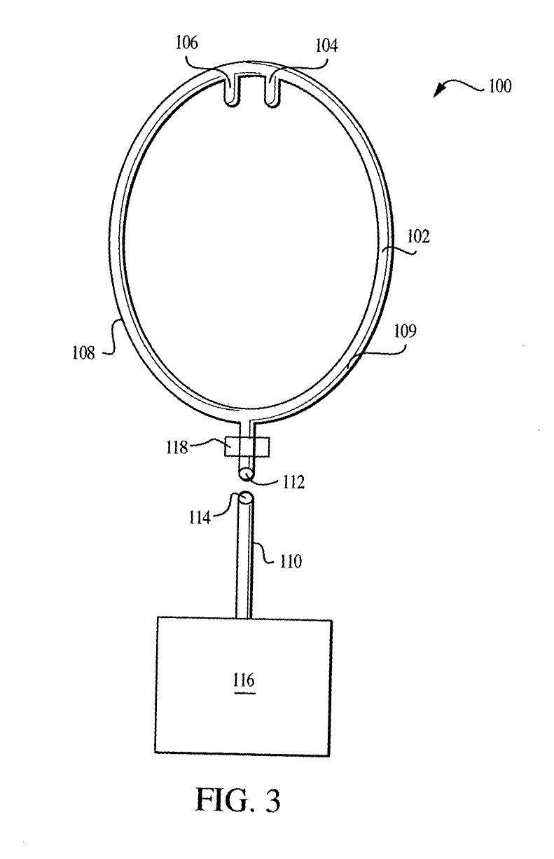

FIG. 3 illustrates an alternate embodiment detachable nasal cannula assembly 100 in accordance with the present invention. The assembly 100 includes a tubular body 102 having a pair of nasal projections 104 and 106 and a main tubing 110. The tubular body 102 includes a nasal tubing segment 108 and a second nasal tubing segment 109. The first nasal tubing segment 108 and the second nasal tubing segment 109 are in communication with each other and preferably form a “loop” or continuous path for the flow of gas or other agents disposed therein. Most preferably, the nasal projection 106 is proximate to and in communication with the first nasal tubing segment 108 and the other nasal projection 104 is proximate to and in communication with the second nasal tubing segment 109. Both tubing segments 108 and 109 are preferably joined to another and are in flow communication with each other at an end 112. The tubular body 102 and main tubing 110 are releasably fastenable by interconnecting portions 112 and 114. The end of the main tubing 110 opposite the main tubing interconnecting portion 114 is releasably fastenable to a device 116 such as an oxygen supply or a carbon dioxide monitor. The assembly 100 further includes a clip 118 that may selectively close the tubing channel of the main tubing 110 or the nasal tubing segment 108 and/or 109.

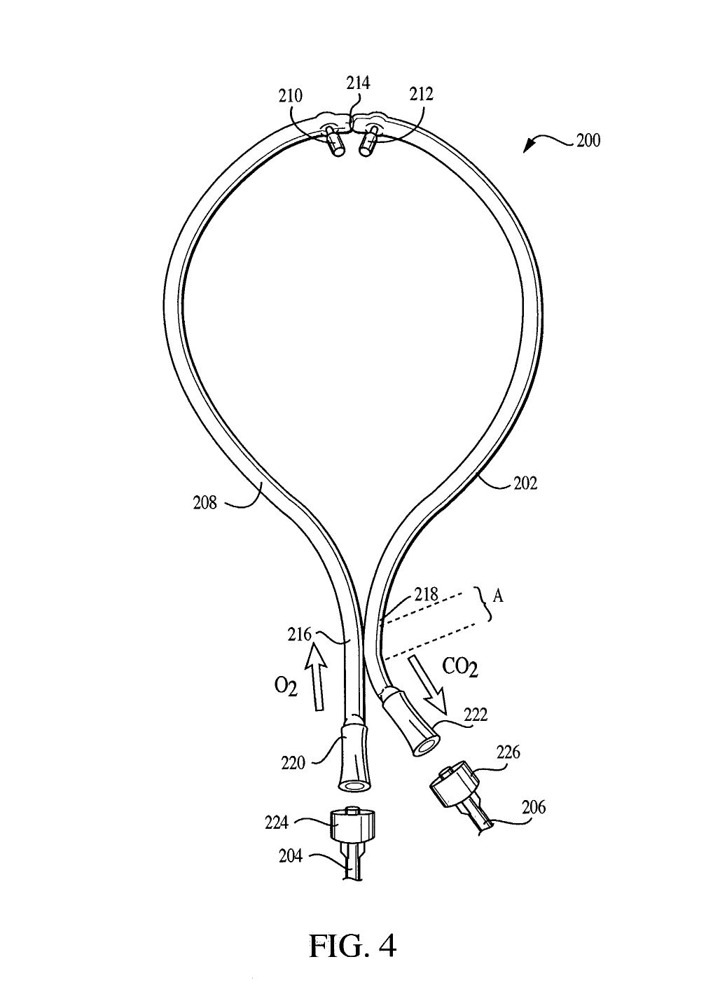

FIG. 4 illustrates another preferred embodiment detachable nasal cannula assembly 200. The assembly 200 includes a nasal cannula portion 202 detached from main tubing 204 and 206. The nasal cannula portion 202 includes a hollow tubular body 208 and has two nasal projections 210 and 212, each extending outwardly and adapted to fit within a corresponding nasal passage of the nose of a patient (not shown). A diaphragm 214 acts as a barrier separating the hollow body 208 of the assembly 200 into an inhalation portion 216, where oxygen is supplied to a patient from an oxygen supply and an exhalation portion 218, where carbon dioxide flows from the patient to a carbon dioxide monitor. Nasal projection 210 acts as an inhalation portion 216 where oxygen is supplied, while nasal projection 212 acts as an exhalation portion 218 where carbon dioxide flows from the patient to a carbon dioxide monitor. FIG. 4 particularly illustrates the flow direction of oxygen and carbon dioxide within the nasal cannula portion 202. Although oxygen and carbon dioxide are depicted as flowing through the tubular body, it will be appreciated that the present invention assemblies are also well suited for use with other gases and materials. The ends of the nasal cannula portion 202 include interconnecting portions 220 and 222. Preferably, the nasal cannula portion 202 is configured such that the body of the tubing extending between the interconnecting portions 220 and 222, is joined to itself at region A as shown in FIG. 4. Preferably, such joining is accomplished by attaching or forming respective outer surface regions of the tubular cannula portion to one another. This configuration promotes the portions 220 and 222 remaining in close proximity with one another. Main tubing portion 204 includes an interconnecting portion 224 that is adaptable to interconnecting portion 220, and main tubing portion 206 includes an interconnecting portion 226 that is adaptable to interconnecting portion 222. The mode of connection in connecting interconnecting portions 220 and 222 with interconnecting portions 224 and 226 include female/male connection, screw fastening, and any other known releasably fastenable mode of connection. The interconnecting portions 220, 222, 224 and 226 may also be in the form of nearly any shape so that the ends of the main tubing 204 and 206 are releasably fastenable with the ends of the nasal cannula portion 202. A closure device similar to clip 36 shown in FIGS. 1 and 2A may also be provided and utilized with the assembly 200.

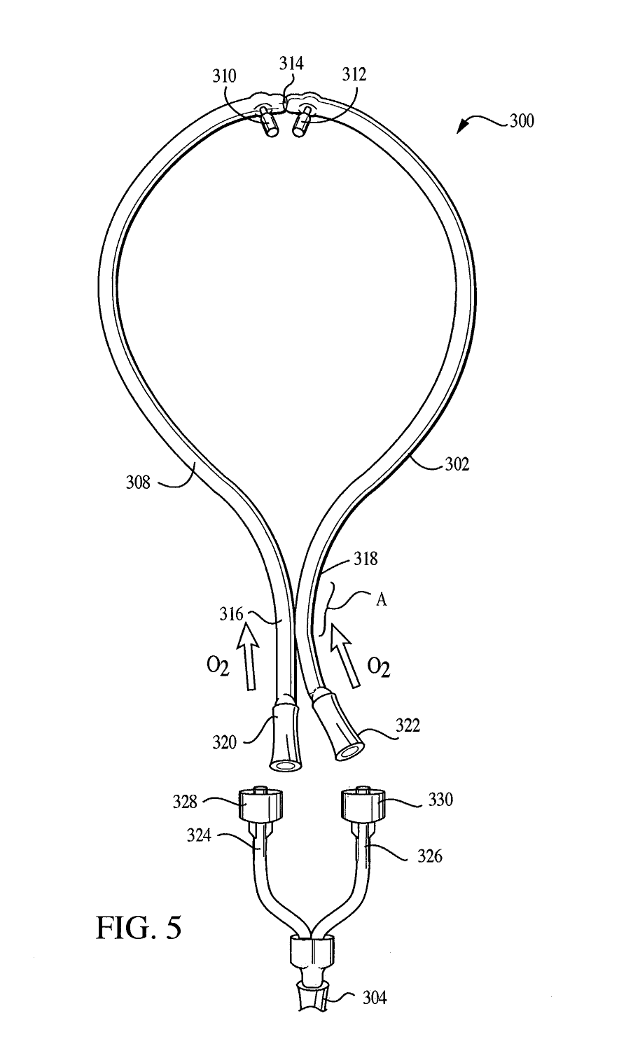

FIG. 5 illustrates another preferred embodiment detachable nasal cannula assembly 300. The assembly 300 includes a nasal cannula portion 302 detached from main tubing 304. The nasal cannula portion 302 includes a hollow tubular body 308 and has two nasal projections 310 and 312, each extending outwardly and adapted to fit within a corresponding nasal passage of the nose of a patient (not shown). A diaphragm 314 acts as a barrier separating the hollow body 308 of the assembly 300. The nasal cannula portion 302 includes two inhalation portions 316 and 318, where oxygen is supplied to a patient from an oxygen supply. FIG. 5 particularly illustrates one possible flow scheme, of oxygen within the nasal cannula portion 302. Although oxygen is depicted, it will be understood that the present invention assemblies are also well suited for use with other gases and materials. The ends of the nasal cannula portion 302 include interconnecting portions 320 and 322. Preferably, the nasal cannula portion 302 is configured such that the body of the tubing extending between the interconnecting portions 320 and 322, is joined to itself at region A as shown in FIG. 5. Preferably, such joining is accomplished by attaching or forming respective outer surface regions of the tubular cannula portion to one another. Main tubing portion 304 includes intermediate tubing portions 324 and 326 attached to an end of the tubing portion 304. The main tubing portion 304 is preferably configured so that the intermediate tubing portions 324 and 326 provide communication, i.e. gas flow, between those portions 324 and 326, and further between those portions 324 and 326 and an opposite distal end of the main tubing portion 304. The end of intermediate tubing portions 324 and 326 include interconnecting portions 328 and 330, respectively. Interconnecting portion 328 is adaptable to interconnecting portion 320. Interconnecting portion 330 is adaptable to interconnecting portion 322. The mode of connection in connecting interconnecting portions 320 and 322 with interconnecting portions 328 and 330 include female/male connection, screw fastening, and any other known releasably fastenable mode of connection. The interconnecting portions 320, 322, 328 and 330 may also be in the form of nearly any shape so that the ends of intermediate tubing 324 and 326 connected to main tubing 304 are releasably fastenable with the ends of the nasal cannula portion 302. A closure device similar to the slip or clamp 36 shown in FIGS. 1 and 2A may also be provided and utilized with the assembly 300.

The present invention nasal cannula assembly is significantly less environmentally adverse than presently available nasal cannulae. Use of the present invention assembly results in less waste material that must be disposed of than currently available cannulae. Less waste results in cost savings and promotes environmentally friendly practices.

Additionally, the present invention nasal cannula assembly is particularly advantageous for applications in which there is no need to monitor carbon dioxide from a patient. In such applications, oxygen may be delivered through the same port that would otherwise be used for monitoring carbon dioxide. This is beneficial in that the patient will receive oxygen through both nostrils. The reasons for this are as follows. If the patient has respiratory problems, or more particularly, has blockage of one of his or her nostrils, administration of oxygen to the patient through only one port could be severely restricted if the blockage occurs in the nasal passage to which the oxygen is being delivered. Use of the present invention nasal cannula assembly delivering oxygen to both nostrils avoids that problem.

It will be appreciated that the present invention includes embodiments utilizing some or all of any of the preferred embodiment cannula assemblies described herein.

Although the preferred embodiments of the present invention have been described in detail, various modifications, alterations and changes or equivalents thereof may be made without departing from the spirit and scope of the invention.

I claim:

1. A detachable nasal cannula assembly comprising:
a tubular nasal cannula portion having a first open end, a second open end opposite from said first end, a first nasal projection providing access to the interior of said tubular portion, a second nasal projection also providing access to the interior of said tubular portion, and a diaphragm disposed between said first nasal projection and said second nasal projection, said diaphragm precluding communication between said first nasal projection and said second nasal projection through said nasal cannula portion;
a first main tubing portion having a first end and a second end opposite from said first end, one of said first end and said second end of said first main tubing portion releasably engageable with said first open end of said nasal cannula portion; and
a second main tubing portion having a first end and a second end opposite from said first end, one of said first end and said second end of said second main tubing portion releasably engageable with said second open end of said nasal cannula portion.

2. The nasal cannula assembly of claim 1 further comprising:
a closure device disposed proximate to at least one of said first open end and said second open end of said nasal cannula portion and adapted to restrict flow of gas flowing through said nasal cannula portion.

3. The nasal cannula assembly of claim 1 wherein a first region of an outer surface of said nasal cannula portion contacts and adjoins a second region of an outer surface of said nasal cannula portion, said first region located proximate said first open end of said nasal cannula portion.

4. The nasal cannula assembly of claim 3 wherein said second region is located proximate said second open end of said nasal cannula portion.

5. The nasal cannula assembly of claim 1 wherein said nasal cannula portion has a length, as measured from said first end to said second end, of from about 15 inches to about 30 inches.

6. The nasal cannula assembly of claim 1 wherein at least one of said first main tubing portion and said second main tubing portion has a length, as measured from said first respective end to said second respective end, of from about 75 inches to about 100 inches.

7. A detachable nasal cannula assembly comprising:
a tubular nasal cannula portion having a first open end, a second open end opposite from said first end, a first nasal projection providing access to the interior of said tubular portion, a second nasal projection also providing access to the interior of said tubular portion, and a diaphragm disposed between said first nasal projection and said second nasal projection, said diaphragm precluding communication between said first nasal projection and said second nasal projection through said nasal cannula portion; and
a main tubing component including a first end having a first interconnecting portion adapted to releasably engage said first open end of said nasal cannula portion and a second interconnecting portion adapted to releasably engage said second open end of said nasal cannula portion, said main tubing component further including a second end opposite from said first end.

8. The nasal cannula assembly of claim 7 further comprising:
a closure device disposed proximate to at least one of said first open end and said second open end of said nasal cannula portion and adapted to restrict flow of gas flowing through said nasal cannula portion.

9. The nasal cannula assembly of claim 7 wherein a first region of an outer surface of said nasal cannula portion is adjoined with a second region of an outer surface of said nasal cannula portion, said first region located proximate said first open end of said nasal cannula portion.

10. The nasal cannula assembly of claim 9 wherein said second region is located proximate said second open end of said nasal cannula portion.

11. The nasal cannula assembly of claim 7 wherein said main tubing component provides communication between said first interconnecting portion and said second end of said main tubing component.

12. The nasal cannula assembly of claim 11 wherein said main tubing component provides communication between said second interconnecting portion and said second end of said main tubing component.

13. The nasal cannula assembly of claim 7 wherein said main tubing component provides communication between said first interconnecting portion and said second interconnecting portion of said main tubing component.

14. A detachable nasal cannula assembly comprising:
a nasal tubular portion including a first nasal projection, a second nasal projection, an interconnecting portion, a first tubular segment extending between said first nasal projection and said interconnecting portion, and a second tubular segment extending between said second nasal projection and said interconnecting portion; and
a main tubing component having a first end and a second end opposite from said first end, said first end adapted to releasably engage said interconnecting portion of said nasal tubular portion.

15. The detachable nasal cannula assembly of claim 14 further comprising:
a closure device disposed proximate to said interconnecting portion of said nasal tubular portion and adapted to restrict flow of gas flowing through said interconnecting portion of said nasal tubular portion.

16. The detachable nasal cannula assembly of claim 14 wherein said nasal tubular portion is in the form of a continuous closed loop such that the first nasal projection is in communication with said interconnecting portion.

17. The detachable nasal cannula assembly of claim 16 wherein said second nasal projection is in communication with said interconnecting portion.

18. The detachable nasal cannula assembly of claim 17 wherein said first nasal projection is in communication with said second nasal projection.
